(19) United States
(12) Patent Application Publication
(10) Pub. No.: US 2025/0002555 A1
(43) Pub. Date: Jan. 2, 2025

(54) METHODS FOR THERAPY AND REHABILITATION OF MOVEMENT DISORDERS

(71) Applicant: City University of Hong Kong, Kowloon (HK)

(72) Inventors: Jufang He, Kowloon Tong (HK); Hao Li, Guangzhou (CN)

(21) Appl. No.: 18/345,430

(22) Filed: Jun. 30, 2023

(51) Int. Cl. C07K 14/705; A61K 31/496; A61K 48/00; A61P 25/14

(52) U.S. Cl. CPC C07K 14/70503; A61K 31/496; A61K 48/0058; A61P 25/14; A61K 38/00

(57) ABSTRACT

A method for treating movement disorders or regaining motor learning ability includes a step of administrating a CCK-B receptor agonist to a subject in need thereof. A CCK-B receptor agonist is used in the manufacture of a medicament for treating movement disorders by administrating it to a subject in need thereof.

### FIELD OF THE INVENTION

The present invention relates to methods for therapy and rehabilitation of movement disorders.

### BACKGROUND

Movement disorder caused by brain injury, such as physical brain injury by accidence, ischemic stroke causing the death of brain neurons, are very common worldwide. Stroke is the second leading cause of death in the world and the first leading cause of death in China, where a fifth population of the world resides. It is one of the main causes of adult disability in many countries. Although stroke mortality is decreasing, the prevalence of people disabled after stroke in China has increased because of the growing and aging population.

Many methods have been invented for motor function restoration for disabled people. Most patents relate to devices facilitating motor training to regain control of the limbs. Motor training alone requires a longer time and is not efficient to help regain movement ability. Drugs focusing on neuronal plasticity or attention have been used together with rehabilitation training. However, available drugs are not very efficient for different patients with various levels of movement disorders and many kinds of side effects limit the usage of the drugs.

Therefore, there remains a need for new therapies for movement disorders.

### SUMMARY OF THE INVENTION

An embodiment of this invention relates to a method for treating movement disorders including a step of administrating a CCK-B receptor agonist to a subject in need thereof.

An embodiment of the present invention relates to a method of regaining motor learning ability including a step of administrating a CCK-B receptor agonist to a subject in need thereof.

An embodiment of the present invention relates to use of a CCK-B receptor agonist in the manufacture of a medicament for treating movement disorders by administrating it to a subject in need thereof.

Without intending to be limited by theory, it is believed that the present invention may provide a novel approach for treating movement disorders by turning on the neuronal plasticity to rescue their motor learning ability. Furthermore, it is believed that the present invention may have very high efficiency and little side effects.

### BRIEF DESCRIPTION OF THE DRAWINGS

FIG. 1A is a schematic picture showing a mouse reaching for a food pellet through a slit;

FIG. 1B shows an exemplary procedure for performing the single pellet reaching task;

FIG. 1C shows the success rate of wildtype (WT) (N=10) and CCK^{-/-} (N=8) mice performing the single pellet reaching task (*p<0.05, **p<0.01, two-way mixed ANOVA, post hoc. comparison between two groups);

FIG. 1D shows representative trajectories of WT and CCK-mice at Day 1, Day 3, and Day 6;

FIG. 1E shows the pairwise Hausdorff distances of the trajectories for comparing the variation in the trajectories of WT (N=10) and CCK^{-/-} mice (N=8), blue and red solid squares represent for average of the Hausdorff distance of WT and CCK^{-/-} mice, respectively;

FIG. 1F shows Hausdorff distance changes with 3-day training of WT and CCK^{-/-} mice;

FIG. 1G is a diagram showing the task phases (reach, grasp, and retrieval) and different reaching results (miss, no-grasp, drop, and success);

FIG. 1H shows detailed reaching results for WT and CCK^{-/-} mice on experimental Day 1 and Day 6 (*p<0.05; paired t-test and t-test);

FIG. 1I shows normalized field Excitatory Postsynaptic Potential (EPSP) amplitude before and after High Frequency Stimulation (HFS) for both WT (N=6, n=21) and CCK^{-/-} mice (N=3, n=7);

FIG. 1J shows the average normalized fEPSP amplitude 10 min before HFS (−10-0 min, before) and 10 min after HFS (50-60 min, after) in the two groups of mice (***p<0.001, N.S. means not significant, two-way mixed ANOVA, pairwise comparison);

FIG. 2A shows an exemplary procedure for studying the effect of local injection of CCKBR antagonist on motor learning;

FIG. 2B shows the success rate of the mice injected with CCKBR antagonist (N=11) and vehicle (N=6), (*p<0.05, ***p<0.001, two-way mixed ANOVA, post hoc. comparison between two groups);

FIG. 2C shows the detailed reaching results, in terms of miss, no-grasp, drop, on Day 1 and Day 5 for mice injected with CCKBR antagonist and vehicle (*p<0.05, paired t-test);

FIG. 3A shows an experiment set-up in which C57BL/6, CCK^{-/-} and C57BL/6 mice injected with CCKBR antagonist are applied for single pellet reaching task training and calcium imaging;

FIG. 3B shows a schematic diagram of calcium imaging;

FIG. 3C shows representative traces of extracted neurons from miniscope using the CNMF-E algorithm, in which the scale bar represents 5 units of the scaled A F/F;

FIG. 3D shows neuronal activity pattern of C57BL/6 (N=10), CCK^{-/-} (N=7) and C57BL/6 mice injected with L365,260 (N=7), in which upper line is from training Day 1 and the bottom is from training Day 6;

FIG. 3E shows neuronal population activities from C57BL/6, CCK^{-/-} and C57BL/6 mice injected with L365,260;

FIG. 3F shows activated population activity (peak activity minus baseline activity) that are calculated for C57BL/6, CCK^{-/-} and C57BL/6 mice injected with L365,260 at Day 1 and Day 6 (*p<0.05, N.S. not significant, Paired t-test);

FIG. 3G shows trial-to-trial population activity correlation at Day 1 and Day 6 for C57BL/6, CCK^{-/-} and C57BL/6 injected with L365,260;

FIG. 3H shows the pairwise Hausdorff distances of the trajectories for C57BL/6, CCK^{-/-} and C57BL/6 injected with L365,260 at Day 1 and Day 6 (*p<0.05, N.S. not significant, one-way RM ANOVA);

FIG. 4A is an image of coronal section showing the virus injection site, where the Cre-dependent AAV-hSyn-DIO-mCherry virus is injected into CCK-Cre mice;

FIG. 4B shows the effective labeling of CCK neuron fibers in the MC;

FIG. 4C shows Cre-dependent retrograde AAV virus injection site in the MC of the CCK-Cre mouse;

FIG. 4D are images of continuous coronal brain sections showing EYFP in the lateral EC, in which the numbers (mm) indicate the position of the sections relative to the bregma;

FIG. 4E are images showing that GAD67 staining does not merge with the retrograde tracking CCK positive neurons in the EC and CaMKII staining merges with the signal of retrograde tracking CCK neurons EC projecting, in which the arrow head indicates the positions of CCK neurons;

FIG. 4F shows percentage of retrogradely labeled neurons merged with CaMKII and GAD67 (n=3);

FIG. 5A shows an experimental paradigm for the chemogenetic experiment, in which Cre-dependent AAV-hSyn-DIO-hM4Di-mCherry or AAV-hSyn-DIO-mCherry is infused into the rhinal cortex of CCK-Cre mice, after four weeks, clozapine or saline is intraperitoneally injected 30 min before training;

FIG. 5B shows the success rate of CCK-Cre mice injected with hM4Di containing virus plus clozapine (hM4Di+clozapine) (N=10) and control virus plus clozapine (mCherry+clozapine) (N=8);

FIG. 5C shows the success rate of CCK-Cre mice injected with hM4Di containing virus plus clozapine (hM4di+clozapine, shared with FIG. 5B) and hM4Di plus saline (hM4Di+saline) (N=11) (*p<0.05, **p<0.01, two-way mixed ANOVA, post hoc. comparison between two groups on different days);

FIG. 6A shows normalized fEPSP amplitude before and after HFS of the MC of CCK^{-/-} mice applied with CCK4 (N=6, n=14) or vehicle (N=6, n=11);

FIG. 6B shows the average normalized fEPSP amplitude 10 min before HFS (−10-0 min, before) and 10 min after HFS (50-60 min, after) in the MC of CCK^{-/-} mice injected with CCK 4 or vehicle (*p<0.05, **p<0.01, two-way mixed ANOVA with Bonferroni pairwise comparison);

FIG. 6C shows an experimental paradigm for CCK rescuing experiment, in which CCK4 or vehicle is injected (i.p.) every day before training;

FIG. 6D shows the success rate of CCK^{-/-} mice injected with CCK4 (N=11) or vehicle (N=10) (*p<0.05, **p<0.01, two-way mixed ANOVA, post hoc. comparison between two groups on Day 5 and Day 6); and

FIG. 6E shows the detailed reaching results for CCK^{-/-} mice injected (i.p.) with vehicle and CCK4 on Day 1 and Day 5 (*p<005, N.S. not significant, paired t-test).

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

Learning to perform motor skills is essential for survival and high quality of life, such as hunting, running, escaping, fighting, playing music, dancing, drawing, and performing an operation. Evidence from electrical stimulation, lesions, imaging, and more targeted manipulation shows that the motor cortex is the center that controls motor behaviors and motor skill learning in the brain. However, it is not completely clear how neuronal plasticity in the motor cortex is regulated.

Brain injury is often caused by ischemic stroke or directly physical damage to the brain. Neurons in the motor cortex required for motor skills and movements die and lose the activity and function to control the performance of dexterous motor skills. Because regeneration ability of neurons in the adult brain is limited, it is difficult for the inactivated brain areas to recover after injury. Brain area compensation is critical for patients to restore the movement ability by relearning the motor skills using compensatory brain areas, such as the opposite hemisphere. Drugs that strengthened the synapses connections could be effective to promote motor skills restoration. However, existing drugs are not very effective to restore movement skills and with severe side effects, as the patients are often aged and the plasticity of the brain is limited, and the drugs may trigger other unwanted neuronal activities.

An embodiment of the present invention relates to a method for treating movement disorders including the step of administrating a CCK-B receptor agonist to a subject in need thereof.

Without intending to be bound by theory, it is believed that the present invention provides a novel approach to treat patients with movement disorders by turning on the neuronal plasticity to rescue their motor learning ability. It is also believed that the method of the present invention is especially beneficial for adults because it enables the plasticity in adulthood and helps in regaining skilled movement ability.

Another embodiment of the present invention relates to a method of regaining motor learning ability including a step of administrating a CCK-B receptor agonist to a subject in need thereof.

In an embodiment herein, the movement disorders may be caused by a condition selected from the group of a brain injury, a stroke, Chorea, Dystonia, Parkinson's disease, brain infarction, neuron death or loss of neuronal plasticity due to aging, and a combination thereof. Without intending to be limited by theory, it is believed that CCK bound to receptors in the postsynaptic membrane directly triggers the neural plasticity by recruiting more receptors (such as AMPA receptor) to the synapses of interest. This would help patients suffering from movement disorders to restore the movement ability.

In an embodiment herein, the CCK-B receptor agonist is selected from the group of CCK4 (Trp-Met-Asp-Phe-NH₂), a CCK4 analogue, and a mixture thereof. The CCK4 analogues may have structures similar to CCK4, and thus we can expect similar functions. It is believed that the main structure of CCK4 (Trp-Met-Asp-Phe-NH₂) is the most critical part, some modifications for C-terminal, N-terminal or both to protect the stability or activity, or replacement of the sulfydryl in Met with other radical without changing the backbone of the molecule may lead to analogues having similar functions as CCK4 itself. In some embodiment, the CCK-B receptor agonist is CCK4. Without intending to be bound by theory, it is believed that CCK4 can pass through the brain-blood barrier, may have fewer side effects and high efficiency with low dosage. In contrast, it is believed that other types of CCK peptides including CCK58, CCK32, and CCK8 cannot pass through the brain-blood barrier.

In an embodiment herein, the CCK-B receptor agonist is administrated via a route selected from the group consisting of intramuscular injection, intraperitoneal administration, intravenous administration, and a combination thereof. In some embodiments, the CCK-B receptor agonist is administrated intraperitoneally. Without intending to be bound by theory, it is believed that intraperitoneal administration (i.p.) can lead to maximum bioavailability. In some embodiments, the CCK-B receptor agonist is administrated intravenously. Without intending to be bound by theory, it is believed that CCK4 can be taken up and carried to the brain via intravenous administration.

In some embodiment herein, the CCK-B receptor agonist is administrated over a period of about 3 days, about 5 days, about 7 days, about 14 days, about 15 days, about 20 days, about 25 days, about 30 days, about 40 days, about 50 days, about 60 days, about 3 months, about 4 months, about 5 months, about 6 months, about 7 months, about 8 months, about 9 months, about 10 months, about 11 months, or about 12 months. In an embodiment herein, the CCK-B receptor agonist is administrated over a period of about one week to four weeks. For example, it can be administrated over a period of about one week, about two weeks, about three weeks, or about four weeks.

In an embodiment herein, the CCK-B receptor agonist is administrated at a dosage of about 0.0036 mg/kg to about 0.036 mg/kg. In some embodiments, the CCK-B receptor agonist is administrated at a dosage of about 0.0036 mg/kg, about 0.0072 mg/kg, about 0.0108 mg/kg, about 0.0144 mg/kg, about 0.018 mg/kg, about 0.0216 mg/kg, about 0.0252 mg/kg, about 0.0288 mg/kg, about 0.0324 mg/kg, about 0.036 mg/kg. In some embodiments, the CCK-B receptor agonist is administrated at a dosage of about 0.036 mg/kg. Without intending to be bound by theory, it is believed that the CCK-B receptor agonist such as CCK4 can be effective in rescuing motor learning ability at such a low dose, and thus the side effects could be minimized.

In an embodiment herein, the CCK-B receptor agonist is administrated in a dosage regimen selected from the group of once daily, twice daily, three times a day, once in 1-8 hrs, once in 1-24 hrs, once in two days, once in three days, once in four days, once in five days, once in six days, once in a week, once in about 8 to about 13 days, once in about two weeks, once in about 15 days to about 30 days.

In an embodiment herein, the CCK-B receptor agonist is administrated at a dosage of about 0.0036 mg/kg to about 0.036 mg/kg every day for a period of about one week, two weeks, three weeks or four weeks.

In an embodiment herein, the CCK-B receptor agonist is effective in regaining motor learning ability in adulthood. Without intending to be bound by theory, it is believed that regeneration ability of neurons in the adult brain is limited, it is difficult for the inactivated brain areas to recover after injury. However, the present invention surprisingly finds that CCK-B receptor agonists can effectively turns on the neuronal plasticity in adults to rescue their motor learning ability.

In an embodiment herein, the stroke is selected from the group of an ischemic stroke, hemorrhagic stroke, cryptogenic stroke, and a combination thereof. In some embodiments, it is believed that the stroke may cause an infarction. Without intending to be limited by theory, it is believed that the CCK-B receptor agonist may work better if the infarct occurs in the cortex only. The reason may be that the infarct brain cortex can be compensated by the adjacent cortex area and CCK-B receptor agonists may help reconstruct the new connections, forming new motor memory.

In an embodiment herein, the brain injury is an injury in motor cortex, which, for example, is a result of physical trauma, or other conditions causing motor cortex damage.

In an embodiment herein, the CCK-B receptor enables neuronal plasticity in motor cortex. In an embodiment herein, the CCK-B receptor is effective in regaining motor learning ability in adulthood. Without intending to be bound by theory, it is believed that the method of the present invention enables the neuronal plasticity in the motor cortex, which controls the body movements. Combined with rehabilitation training, it helps the disordered limbs regain the skilled moving function.

An embodiment of the present invention relates to use of a CCK-B receptor agonist in the manufacture of a medicament for treating movement disorders by administrating it to a subject in need thereof.

Without intending to be bound by theory, it is believed that it is the first time to report the use of the CCK-B receptor agonist for the treatment of movement disorders. The present invention provides a new approach for regaining the motor learning ability.

In an embodiment herein, the CCK-B receptor agonist is in a dosage form suitable for an administration route selected from the group of intramuscular injection, intraperitoneal administration, intravenous administration, and a combination thereof. In some embodiments, the CCK-B receptor agonist is in a dosage form suitable for intraperitoneal administration. Without intending to be bound by theory, it is believed that intraperitoneal administration (i.p.) can lead to maximum bioavailability. In some embodiments, the CCK-B receptor agonist is in a dosage form suitable for intravenous administration. Without intending to be bound by theory, it is believed that CCK4 can be taken up and carried to the brain via intravenous administration.

In an embodiment herein, the CCK-B receptor agonist is comprised in an amount of about 80% to about 90% by weight, for example, about 82%, about 85%, about 87% by weight in the medicament.

### EXAMPLES

### Materials and Methods

### Animal

Young adult wildtype (C57BL/6) mice and C57BL/6 background transgenic mice, CCK-Cre (CCK-ires-Cre, Strain Cat #012706, Jackson Laboratory, https://www.jax.org/strain/012706, USA) and mice with a knockout of cck gene (CCK^{-/-}) (CCK− CreER, Strain Cat #012710, Jackson Laboratory, https://www.jax.org/strain/012710, USA), are used for behavior, electrophysiology and anatomy experiments. All mice are housed in the pathogen-free 12 hours light/dark cycle holding room with the temperature at 20-24° C. All experimental procedures are approved by the Animal Subjects Ethics Sub-Committee of the City University of Hong Kong.

### Single Pellet Reaching Task

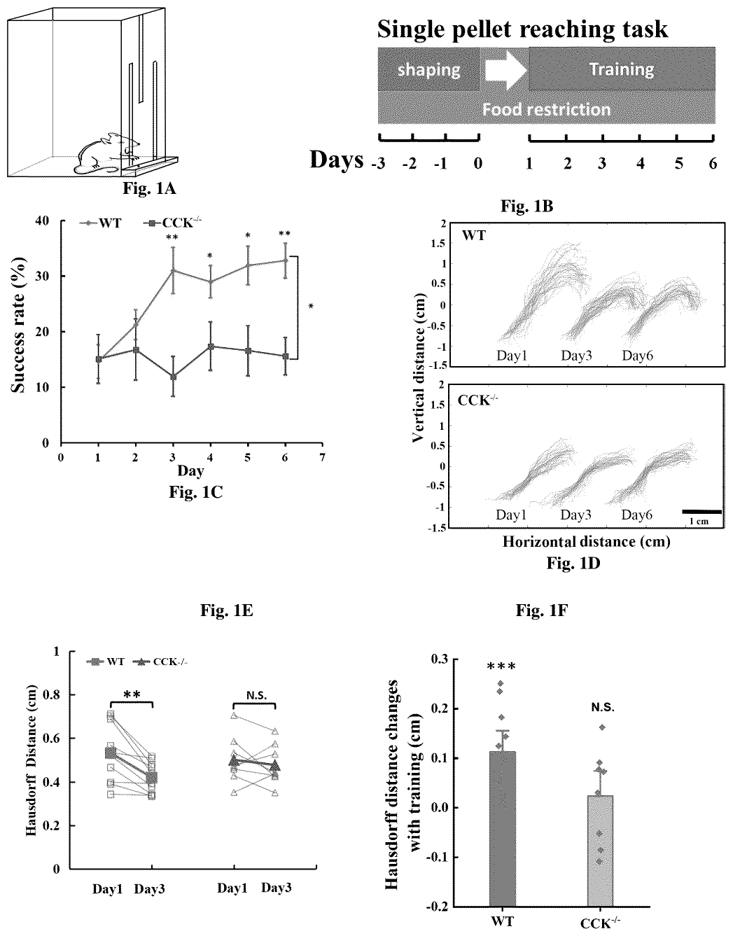

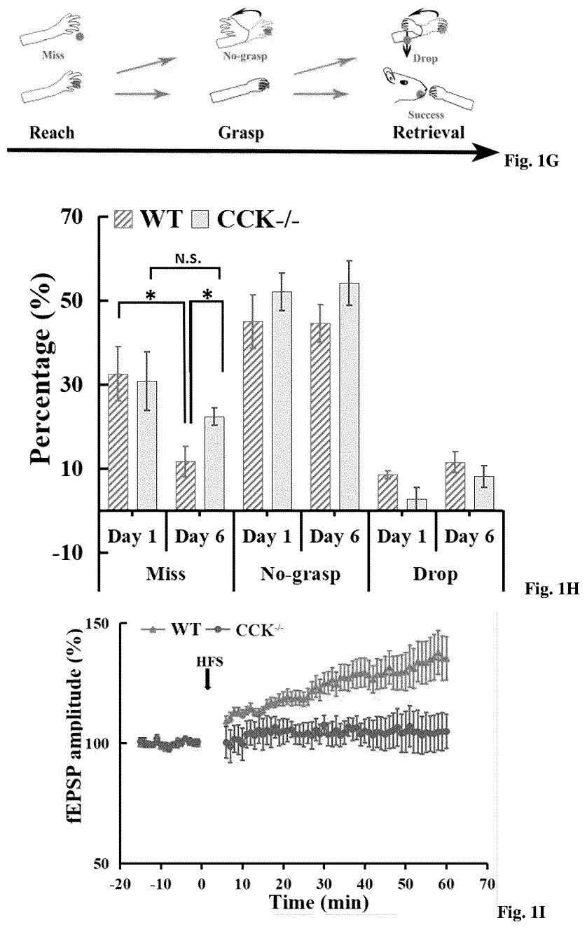

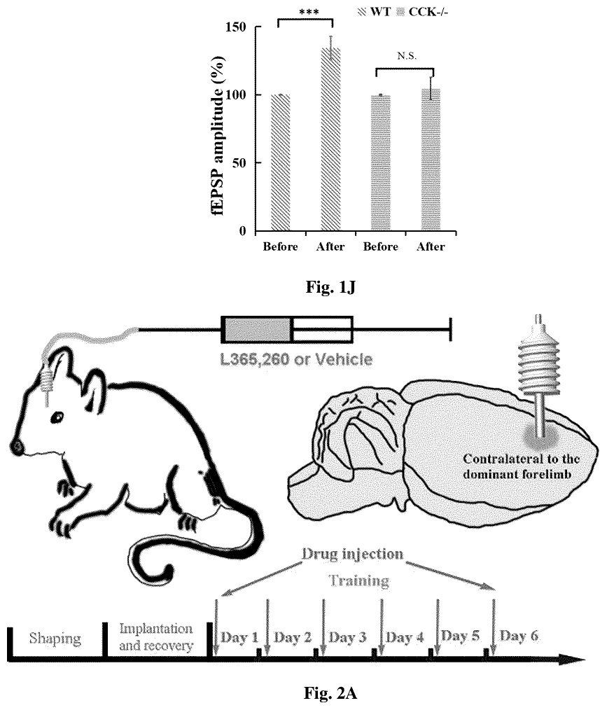

The behavioral experiment, single pellet reaching task, is modified based on a previously established procedure^{1,2}. A clear and transparent Plexiglas chamber (5 mm thickness, dimensions 20 cm×15 cm×8.5 cm) is built for mice training, with three 5 mm wide slits on the front wall; one is in the middle, the other two are 1.9 cm to the side, respectively. A 1.0-cm-height exterior shelf is affixed in front of the front wall to hold the chocolate pellets (Cat #1811223, 20 mg, TestDiet, https://www.testdiet.com/, USA) for reward. The food pellet is placed 0.7 cm away from the front wall and 0.4 cm away from the midline of the slit, to encourage the mouse to use the dominant hand for catching (FIG. 1A). The task has two periods, shaping and training. Mice are food restricted to keep approximately 90% body weight of the original weight during the whole process (FIG. 1B). Three days before training, the mouse is placed in the chamber and allowed to acclimate to the environment and determine the dominant hand. On shaping day one, two mice from the same cage are placed into the chamber for 20 min to acclimate to the environment; on shaping day two, an individual mouse is placed into the chamber for 20 min. During shaping, 10 food pellets are fed for each mouse every day to train mice eating food pellets. On shaping day 3, a food tray full of food pellets is placed in front of the middle slit. The mouse can get the food reward by catching it through the slit with either hand. The experiment stops when 20 times of reaching attempts are finished for each mouse. The dominant hand should be the one that shows over 70% preference. During the training period, mice reach for food pellets through the slit by the dominant hand, 40 attempts within 20 min every day. Only attempts by the dominant hand are counted. Based on the results of the attempts, the reaching attempts show four types: miss, no-grasp, drop, and success. A “miss” means that the mouse's hand does not touch the food pellet. A “no-grasp” means that the mouse's hand touches the food pellet, but it does not successfully grasp the pellet. A “drop” represents the mouse grasps the pellet, but it dropped due to whatever reasons during the retrieval. A “success” is a reach in which the mouse successfully retrieves the pellet and put it into their mouth. A high-speed camera is placed on the side of the chamber to videotape the mice's reaching behavior at 60 frames per second. The success rate is calculated as the number of successful attempts/the total attempts. The miss rate, the no-grasp rate, and the drop rate are also calculated to evaluate the mice's performance of each step. Hausdorff distances, the greatest of all the distances from a point in one set to the closest point in the other set, are calculated to assess the variation of trajectories.

### CCKBR Antagonist Injection

C57BL/6 mice are implanted a cannula in the motor cortex (coordinates: AP, 1.4 mm, ML, −/+1.6 mm, DV, 0.2 mm) contralateral to the dominant hand of the mice, followed by three days of recovery. Mice are grouped into antagonist and vehicle groups. L365,260 (CCKBR antagonist) (1 μl, 20 μM, Cat #2767, Bio-Techne, https://www.bio-techne.com/, USA) or vehicle (0.1% DMSO dissolved in ACSF) is injected into the motor cortex through the cannula with the flow rate of 100 nl/min pumped by a syringe pump (Hamilton, http://hamiltoninstrument.com, USA), before the mice are placed into the chamber for the single pellet reaching task training.

### Chemogenetic Manipulation

A Chemogenetic experiment is performed on CCK-ires-Cre mice (Cat #012706, Jackson Laboratories, https://www.jax.org/strain/012706, USA). Cre-dependent hM4Di virus is injected into the rhinal cortex. Detailed coordinates and volumes are described in the virus injection part. Mice are used for single pellet reaching task training four weeks post virus injection. Thirty minutes before behavior training, clozapine (0.4 mg/kg, Sigma-Aldrich, https://www.sigmaaldrich.com, USA, dissolved with 0.1% DMSO) is intraperitoneally injected to inactivate the activity of the CCK-expressing neurons in the rhinal cortex. The same volume of vehicle (0.9% saline solution with 0.1% DMSO) is injected for the sham control group. A negative control virus (AAV8-hSyn-DIO-mCherry) combined with intraperitoneal clozapine injection is also carried out to exclude the influence of clozapine on motor learning ability.

### Virus Injection and Surgical Process

AAV8-hSyn-DIO-mchery, AAV8-hSyn-DIO-hM4Di-mCherry are diluted to the titer around 5×10¹² copies/ml and AAVᵣₑₜᵣₒ-EF1a-DIO-EYFP, and AAV-hSyn-CaMKII-GCaMP6s-SV40 are diluted to the titer around 1×10¹³ copies/ml and injected into the mouse cortex as previously described^{3,4}. The mice are anesthetized with pentobarbital with their fur between two ears trimmed and fixed on a stereotactic apparatus (RWD, China). Firstly, the head skin of the mouse is cleaned and sterilized with 70% alcohol and removed to expose the skull totally. To accurately locate the areas of interest, the head is adjusted between middle and lateral, and anterior and posterior. In order to completely inactivate the rhinal cortex, two injection sites per hemisphere are determined for virus injection using the following coordinates: site 1: anteroposterior (AP), −3.52 mm from Bregma, mediolateral (ML), 3.57 mm, dorsoventral (DV), −3.33 mm from the brain surface; site 2: AP, −4.24 mm from Bregma, ML, 3.55 mm, DV, −2.85 from the brain surface. Microinjections are carried out using a microinjector (World Precision Instruments, https://www.wpiinc.com, USA) and a glass pipette (#504949, World Precision Instruments, https://www.wpiinc.com, USA). The volume is 200 nl for each site and the flow rate is 50 nl/min.

To track the projection of CCK neuron from the rhinal cortex to the motor cortex, retrograde AAV-EF1a-DIO-eYFP is injected into the motor cortex. The coordinates is: site 1: AP, 1.8 mm to the Bregma, ML, 1.2 mm, DV 200 μm, and 600 μm; site 2: AP, 1.0 mm to the Bregma, ML, 1.5 mm, DV, 200 μm and 600 μm. The volume of each site at each DV is 200 nl and the flow rate is 20 nl/min to protect the fluid from flowing out. An anterograde AAV-hSyn-DIO-mCherry is also used for projection tracking by injecting the virus into the rhinal cortex of the hemisphere. The specific coordinates are as described above. After virus injection, skins are seamed with sterilized sewing thread, and the cut is spread with antibiotic paste to protect it from pathogens and accelerate healing.

The surface virus infusion process for the calcium imaging is performed as described previously with mild modification⁵. A wide-tip glass pipette is prepared by a micropipette puller and then cut, polished, and flame-treated to make it even and smooth. Mice are intraperitoneally injected with dexamethasone (0.2 mg/kg, s.c.) and carprofen (5 mg/kg, s.c.) to protect the brain from swelling and inflammation. Three hours later, mice are anesthetized with pentobarbital. The periosteum covered on the skull is removed, cleaned, and dried with 100% alcohol to prevent the skull and tissues from growing. A 3×3 mm² window above the motor cortex contralateral to the dominant hand is opened with a hand drill, and the bone debris is carefully removed with fine forceps. After that, the dura around the injection area is removed (open a dura hole of about 1 mm²) to expose the pial tissue for virus infusion. The tip of the pipette tightly covered the brain surface by lowering 400-500 μm, and 0.6 μl virus is infused at the speed of 0.06 μl/min. A 3×3 cover glass (thickness, around 150 μm) is attached to the brain surface, and gentle pressure is applied to keep the cover glass at the level same as the skull. The edge of the cover glass is sealed with superglue. After the glue is totally hardened, the skin is stretched back and sutured.

### Baseplate Implantation

2-3 weeks after cranial window implantation, the scalp over the skull is totally removed with surgical scissors. Success implantation shows a clear observation window without blood on the brain surface and a cover glass tightly fixed on the skull. The cover glass surface is gently cleaned with Ringer's solution and lens paper, and the regrowth of periosteum on the skull is removed with fine forceps. Before baseplate implantation, the skull is dried with 100% alcohol, covered with Metbondglue, and a thick layer of dental acrylic except for the cover glass for observation.

A one-photon miniscope (UCLA miniscope V4, Lab maker, https://www.labmaker.org/products/miniscope-v4, Germany) connected to the data acquisition software is attached to the baseplate, secured on the stereotaxic micromanipulator, and gradually lowered to the cover glass until there is only a 1 mm gap between the skull and the baseplate. The LED is turned on and the focal distance of the electrowetting lens is adjusted to 0 on the software. The position of the miniscope is adjusted until the brain tissue is observed in the data acquisition system. Dental acrylic is used to fix the baseplate to the acrylic cap covering the skull around the window. Once the dental acrylic has hardened, the miniscope is removed, and a metal cap is attached to the baseplate to protect the cover glass window.

### Calcium Imaging and Fluorescent Signal Analysis

After the implantation of the baseplate, a miniscope model is attached to the baseplate, and the mouse is placed in the chamber to acclimate to the weight of the miniscope for 20 minutes for 3 days. The LED laser and focal plate are slightly adjusted until the cells with fluorescent protruded from the background. A web camera is also connected to the data acquisition software and recorded the behavior movement of the animal simultaneously. An imaging field of about 1.0×1.0 mm² (resolution: 608×608 pixels) video at approximately 10 min long is recorded. To clearly figure out the role of CCK played in the neuronal plasticity of the motor cortex from CCK^{-/-} mice, C57BL/6 mice as well as C57BL/6 mice that intraperitoneal injection of CCKBR antagonist, L365,260 (0.4 mg/kg, Cat #2767, Bio-Techne, https://www.bio-techne.com, USA). Raw AVI videos are firstly spatially down-sample by two folds to reduce the size of the videos by Fiji (Image J, https://imagej.net/imagej-wiki-static/Fiji/Downloads, USA). Then a MATLAB algorithm, NoRMCorre, is applied for piecewise rigid motion correction before data analysis. The calcium signals are extracted with the MATLAB code of Constrained Nonnegative Matrix Factorization for microEndoscopic (CNMF-E) (code availability: https://github.com/zhoupc/CNMF_E) 6. The scaled fluorescent calcium signal overtime is extracted as C_raw. The raw data is then deconvolved. The activity higher than 3 times the standard deviation of baseline fluctuation is deemed as a calcium event which has been revealed to be associated with neuronal spiking activity, and the rising phase of which is searched and used for further neuronal activity analysis^{7,8}. Timestamps from both the behavior videos and the calcium imaging videos are aligned to find out the time window when the mouse perform the reaching task. Neuronal activity in the time window from 100 ms before reaching to 100 ms after retrieval is considered the activity related to the movements. Wilcoxon ranksum test is conducted between activity inside the time window and activity outside (p<0.05) to exclude the neurons that activate indiscriminately or not correlate with the reaching task. Neurons with the average activity in the time window higher than the average outside the time window are considered movement-related neurons. The neurons are aligned based on each neuron's sorted time of peak event to visualize each and all the neuronal activity patterns during the reaching task. The recurrence of neuronal activities related to the movements is also elevated by pairwise comparison of the population neuronal activity between trials using the Pearson correlation coefficient.

### Immunohistochemistry

Four weeks after virus injection, mice are perfused with 50 mL cold PBS buffer (1×) to remove the blood and 50 mL 4% paraformaldehyde (PFA) in PBS to fix the brain tissue. The skull is carefully opened, and the brain is removed from the skull and fixed by immersing it in 4% PFA at 4° C. for 24 hours, then dehydrated in 30% sucrose PBS solution until it sinks to the bottom. Brains are covered with OTC, freezing fixed, and sectioned to a thickness of 50 μm using a freezing microtome (Leica, https://www.leica.com, Germany). Brain slices are preserved in an anti-freezing solution (25% glycerol and 30% ethylene glycol, in PBS) and stored in the −80° C. refrigerator.

For immunostaining, the brain slices are washed 3 times using 1×PBS in a shaker and incubated in blocking solution (10% normal goat serum and 0.2% TritonX-100 in PBS) for more than 1.5 hours in a shaker and incubated with the primary antibody (Cat #MAB5406, Mouse anti-GAD67, Millipore, https://www.emdmillipore.com, USA; Cat #Ab22609, Mouse anti-CaMK2a, Abcam, https://securedrtest.abcam.com, UK; Cat #M11217, Rabbit anti-mCherry, Invitrogen, https://www.fishersci.com/, USA) in 0.2% Triton and 5% Goat serum in PBS at 4° C. for 24-36 hours. Slices are washed with PBS four times before incubating with the second antibody (Cat #115-585-003, Alexa Fluor 594 conjugated goat anti-mouse, Alexa Fluor 594 conjugated goat anti-rabbit, Jackson ImmunoResearch, https://www.jacksonimmuno.com, USA) diluted in 0.1% Triton PBS solution for 3 hours. Finally, slices are washed in 1×PBS 4 times, then incubated with DAPI (1 mg/ml) for 10 min, mounted on slides and sealed with mounting medium (70% glycerol inPBS). Slices are observed and imaged with a confocal laser-scanning microscope (Zeiss, https://www.zeiss.com, Germany) using 10× and 20× air objectives or 40× and 60× oil immersion objectives.

### Brain Slice Electrophysiology

The slice electrophysiology experiment is carried out following the methods reported previously⁹. In the experiments, 6-8 weeks old C57BL/6 or CCK^{-/-} mice are anesthetized with isoflurane in a small chamber. The mouse head is cut, and the brain is rapidly removed and put into an oxygenated (95% O₂-5% CO₂) artificial cerebral spinal fluid (ACSF) cold bath containing 26 mM of NaHCO₃, 2 mM of CaCl₂), 1.25 mM of KH₂PO₄, 1.25 mM of MgSO₄, 124 mM of NaCl, 3 mM of KCl and 10 mM of glucose, pH 7.35-7.45. The brain is sectioned from the middle line into two hemispheres. The portions with the brain areas of the motor cortex are trimmed and glued on the ice-cold stage of a vibrating tissue slicer (VT1000S, Leica, https://www.gmi-inc.com/product/leica-vt1000s-microtome, Germany). Coronal sections of slices containing the motor cortex (300 μm thick) are trimmed and gently transferred into an ACSF containing chamber, which is put in a water bath at 28° C. and oxygen blowing continuously. After 2 hours of recovery in the ACSF bath, the slice is applied for the following electrophysiological recording.

A commercial 4-slice 8×8 channels recording system (MED, Panasonic Alpha MED Sciences, https://alphamedsci.com/english/company, Japan) is applied to record the fEPSPs. The MED probe is composed of 64 microelectrodes; the distance between the two channels is 50×50 μm, (MED-P515A, 64-channel, 8×8 pattern, 50×50 μm, inter-electrode distance 150 μm or MED-PG515A).

After recovery, the motor cortex slice is covered by the recording electrodes. A fine-mesh anchor (Warner Instruments, Harvard Bioscience, https://www.harvardbioscience.com, USA) is covered on the brain slice to stabilize it, and the probe chamber is perfused with fresh ACSF oxygenated with oxygen with a peristaltic pump (Minipuls 3, Gilson, https://us.vwr.com/store/product/29028243/minipuls-3-peristaltic-pumps-gilson, USA), and the water bath is kept at 32° C. After 20 min of recovery, one of the microelectrodes in the area of interest is selected as the stimulating electrode through an inverted camera (DP70, Olympus, https://www.olympus-lifescience.com, Japan). The surface layer of the motor cortex is stimulated with constant current pulses at 0.1 ms in duration at 0.017 Hz by the connected controlling software, data acquisition software (Mobius, Panasonic Alpha MED Sciences, https://alphamedsci.com/english/company, Japan). After the baseline recording, which is stimulated at the currency of that triggering around 50% of the saturating potential. For drug application, CCK4 (final concentration: 500 nM) or vehicle is injected into the electrode dishes. High-frequency stimulation (HFS) (25 bursts at 120 Hz for each burst, at the highest intensity) is applied to the simulation probe. The electrophysiological data are extracted and analyzed with offline software, Mobius software. For quantification of the LTP data, the initial amplitudes of fEPSPs are normalized and expressed as percentage changes over the averaged baseline activity. The fEPSP is normalized based on the percentage of the baseline potential.

Rescue of the Motor Learning Ability of the CCK^{-/-} Mice with CCK4

CCK4, a tetrapeptide derived from the peptide of CCK is selected as a potential drug to rescue the motor learning defect caused by the lack of CCK, because CCK4 remains the function to activate the CCK receptor but has a much smaller molecule than CCK8s or CCK58, making it transmit through the brain-blood barrier easily and smoothly¹⁰⁻¹¹. Therefore, intraperitoneal injection of the CCK4 is a simple and easily available way to rescue CCK lack caused motor learning defects. After shaping, CCK^{-/-} mice are injected intraperitoneally with CCK4 (0.45 mg/kg, Cat #ab141328, Abcam, https://securedrtest.abcam.com, UK) or vehicle before training every day.

### Statistical Analysis

Group data are shown as mean±SEM (standard error of the mean) unless otherwise stated. Statistical analyses, including paired t-tests, one-way RM ANOVA, and two-way mixed ANOVA, are conducted in SPSS 26 (IBM, Armonk, NY, https://www.ibm.com, USA). Statistical significance is defined as p<0.05 by default.

### Results

### The Role of CCK in Motor Learning

A previous study shows that CCK is a key factor regulating neuronal plasticity that enhances long-term memory formation in the auditory cortex⁹. Therefore, a single pellet reaching task is adopted to train transgenic CCK^{-/-} mice and their wildtype control (C57BL/6) to use the dominant forelimbs and obtain food rewards as the method to determine whether CCK is involved in motor learning (FIG. 1A). This task, including shaping and training, has been adopted in many studies on motor skill learning and motor control systems, especially those controlling the forelimb (FIG. 1B)^{1,8}. The performance of wildtype and CCK^{-/-} mice is evaluated based on the success rate, which requires accurate performance in aiming, reaching, grasping, and retrieval. The success rate of CCK^{-/-} mice does not increase after six days of training, remaining at the baseline level of approximately 15% (FIG. 1C, CCK^{-/-} mice, one-way RM ANOVA, F[5,35]=0.574; p=0.72; post hoc. pairwise comparison between different days, Day 1 vs. Day 3, 15.05%±4.40% vs. 11.91%±3.60%, p=0.59; Day 1 vs. Day 6, 15.05%±4.40% vs. 15.59%±3.36%, p=0.924), while wildtype mice perform much better, of which the success rate increases significantly to 30.94% on day 3 and remains at a plateau until the end of training (FIG. 1C; WT mice, one-way RM ANOVA, F[5,45]=4.904; p<0.001; post hoc. pairwise comparison, Day 1 vs. Day 3, 14.63%±3.05% vs 30.94%±4.17%, p=0.013<0.05; Day 1 vs Day 6, 14.6%=3.05% vs. 32.76%±3.12%, p=0.004<0.01; between WT and CCK^{-/-} mice, two-way mixed ANOVA, significant interaction, F[5,80]=4.03, p=0.003<0.01; post hoc. comparison between two groups, F[1,16]=7.697, p=0.014<0.05; WT vs. CCK^{-/-}, Day 3, 30.94%±4.17% vs. 11.91%±3.60%, F[1,16]=11.239, p=0.004<0.01; Day 4, 28.96%±2.90% vs. 17.37%±4.35%, F[1,16]=5.266, p=0.036<0.05; Day 5, 31.90%±3.50% vs. 16.56%±4.51%, F[1,16]=7.465, p=0.015<0.05; Day 6, 32.76%±3.12% vs. 15.59%±3.36%, F[1,16]=13.906, p=0.0018<0.01). The success rates of wildtype and CCK^{-/-} mice are similar on day one, indicating that CCK does not affect the basic ability to carry out the task, although the learning ability is inhibited (FIG. 1C; t-test, WT vs. CCK^{-/-}, 14.62%=3.05% vs. 15.05%±4.40%, p=0.9366). The variation of trajectories of the hand movement is also evaluated. The deviation of the trajectories of different trials of a wildtype mouse became visibly smaller on Day 3 compared with that on Day 1, while that of a CCK^{-/-} mouse shows no visible improvement (FIG. 1D). The Hausdorff distances, the greatest of all the distances from a point in one set to the closest point in the other set, are calculated to evaluate the variation of trajectories¹². The Hausdorff distance for the trajectories of wildtype and CCK^{-/-} mice are similar at Day 1 (FIG. 1E and FIG. 1F; t-test, WT vs. CCK^{-/-}, 0.53±0.04 cm vs. 0.50±0.04 cm, p=0.5908). However, after 3 days' training, the Hausdorff distance for wildtype mice significantly decreases while CCK^{-/-} mice remains unchanged (FIG. 1E and FIG. 1F; paired t-test, WT, Day 1 vs. Day 3, 0.53±0.04 cm vs. 0.42±0.02 cm, p=0.003<0.01; CCK^{-/-}, Day 1 vs. Day 3, 0.50±0.04 cm vs. 0.48±0.03 cm, p=0.514).

Failures in retrieving the pellets, including miss, no-grasp, and dropping, are also applied to assess specific learning defects in different movement phases of the complex task, comprising the deficiency of “success”, which only indicates the final execution results (FIG. 1G). “Miss”, representing no touching of the food pellet in front of the wall of the chamber, is due to inaccurate aiming and inadequate preparation of the neuronal system, especially processes involved in motor control and execution. A “no-grasp” is a reach in which the mouse shows a defect in finger closure around food pellets for retrieval. A “drop” is a reach in which the mouse drops the food pellet before putting it into the mouth, although the pellet is grasped correctly, indicating a defect in neurons controlling the retrieval process. The miss rate of CCK^{-/-} mice is higher than that of wildtype mice, suggesting that CCK may affect the learning ability in aiming and preparing to execute a motor task (FIG. 1H; paired t-test, WT, Day 1 vs. Day 6, 32.54%±6.43% vs. 11.62%±3.58%, p=0.0127<0.05; CCK^{-/-}, Day 1 vs. Day 6, 30.77%±7.07% vs. 22.25%±2.09%, p=0.1732; t-test, WT vs. CCK^{-/-}, Day 6, 11.62%±3.58% vs. 22.25%±2.09%, p=0.0265<0.05).

Further, an electrophysiology experiment is performed on the slices of the motor cortex from wildtype and CCK^{-/-} mice to investigate the potential physiological causes for the defects in motor skill learning of CCK^{-/-} mice. LTP in field excitatory postsynaptic potential (fEPSP) after HFS in the wildtype mice is observed, but there is no LTP from CCK^{-/-} mice, suggesting that CCK plays a key role in neuronal plasticity in the motor cortex (FIG. 1I, FIG. 1J; two-way mixed ANOVA, F[1,24]=3.154, p=0.088; post hoc. pairwise comparison, WT, before vs. after HFS, 100.06%±0.35% vs. 134.38%±8.61%, F[1,20]=17.255, p<0.001; CCK^{-/-}, before vs. after, 99.82%±0.48% vs. 104.62%±7.99%, F[1,6]=0.5, p=0.506).

In summary, CCK mice show an impaired ability in motor skill learning in the single pellet reaching task and a defect in the LTP induction in the motor cortex.

### A CCKBR Antagonist Injection in the Motor Cortex Inhibited the Motor Learning Ability of C57BL/6 Mice

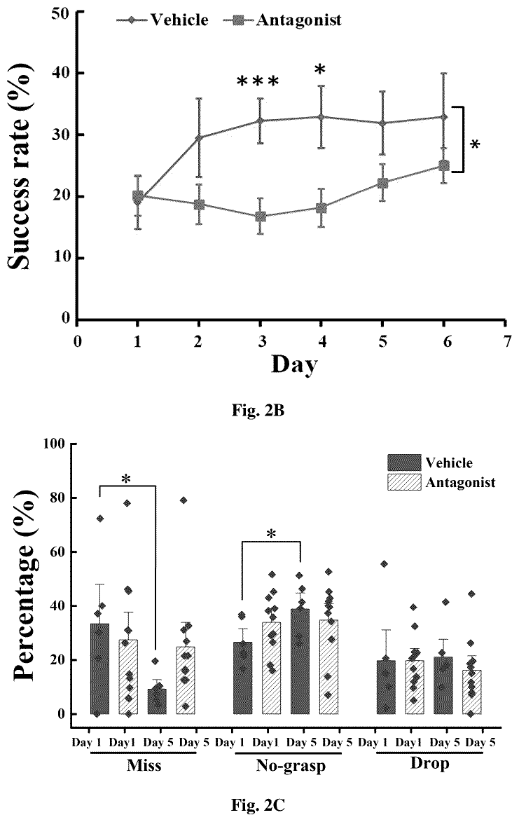

As deletion of the cck gene in the CCK^{-/-} is general, the above experiment results could not indicate the source of CCK and their action site in the brain. The manipulation of the CCK signaling is limited in the motor cortex, targeting its primary receptor, CCKBR, in the neocortex. A drug infusion cannula is implanted into the motor cortex contralaterally to its dominant forelimb and the CCKBR antagonist, L365,260 or its vehicle control is injected to examine whether blocking the CCKBRs in the motor cortex could affect motor skill learning (FIG. 2A). L365.260 is infused to the experimental group or vehicle (ACSF+0.1% DMSO) is infused to the control group through the implanted drug cannula in the motor cortex every day before training. The success rate of pellet retrieval of the experimental group is not improved through the 6-day training period (FIG. 2B, one-way RMANOVA, F[5,50]=1.959, p=0.101), while that of the vehicle control group is significantly improved to 32.30% at Day 3 and kept at this level till the end of training (FIG. 2B, one-way RM ANOVA, pairwise comparison, Day 1 vs. Day3, 19.02%±4.27% vs. 32.30%±3.62%, F[1,10]=5.628, p=0.039<0.05; Day 3 vs. Day 6, 32.30%±3.62% vs. 32.90%±7.07%, F[1,10]=0.007; p=0937). The differences in the success rate between the experimental and control groups are significant (Two-way mixed ANOVA, F[5,70]=1.881, p=0.109; post hoc. comparison between Antagonist and Vehicle, F[1,14]=5.066, p=0.041; Day 3, Antagonist vs. Vehicle, 16.80±2.83% vs. 32.30±3.62%, F[1,15]=11.266, p=0.0048<0.01; Day 4, 18.16±3.12% vs. 32.90±5.03%, F[1,15]=6.876, p=0.019<0.05). This result suggests that CCK participates in motor skill learning by regulating neuronal plasticity in the motor cortex.

For the detailed reaching results, the performance of the experimental and control groups on Day 1 and Day 5 are compared. The number of “miss” of the antagonist group has no significant decrease with learning, but for the vehicle group, it drops from 35% to 10%, indicating that the aiming and advance learning abilities are significantly impaired by inactivation CCKBRs in the motor cortex (FIG. 2C, paired t-test, Antagonist, Day 1 vs. Day 5, 27.34%±9.85% vs. 24.75%±2.34%, p=0.794; Vehicle, Day 1 vs. Day 5, 33.05%±6.68% vs. 9.17%±6.04%, p=0.044<0.05). For the “no-grasp” outcome, the vehicle group increases significantly by 12.24%, indicating that the implantation of a cannula may cause injury to the motor cortex, leading to defects in digit learning (FIG. 2C; paired t-test, “no-grasp”, Day 1 vs. Day 5, 26.49%±3.26% vs. 38.73%±4.05%, p=0.017<0.05), while that of antagonist shows no improvement increase (Paired t-test, “no-grasp”, Day 1 vs. Day 5, 33.78%=3.36% vs. 34.69%±4.12%, p=0.85). The drop rates of both groups have no significant changes, indicating that the retrieval learning ability is not affected (FIG. 2C). In summary, CCK plays a critical role in memory acquisition by activating the CCK receptors in the motor cortex at the overall level.

### Calcium Imaging of Layer 2/3 of the Motor Cortex During Motor Skill Learning

Based on the outcome of the above drug infusion experiment and previous studies, the motor cortex is one of the primary sites for motor skill learning⁸. Previous studies find that neuronal activity patterns in the Layer 2/3 of the motor cortex are refined, exhibiting reproducible spatiotemporal sequences of activities with motor learning⁷. Therefore, calcium imaging of neurons in the motor cortex layer 2/3 of C57BL/6 mice, CCK^{-/-} mice and C57BL/6 mice injected with the CCKBR antagonist is performed to determine the activities of neurons in the motor cortex during the single pellet reaching task.

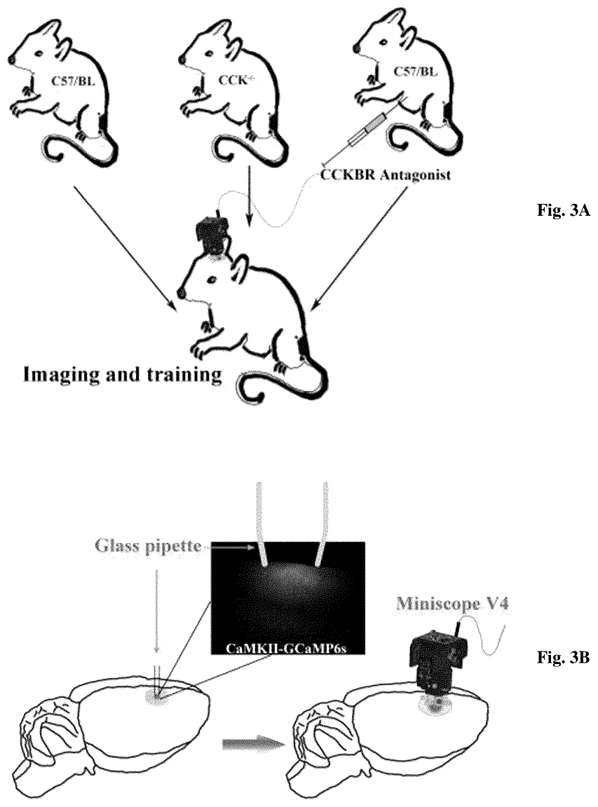

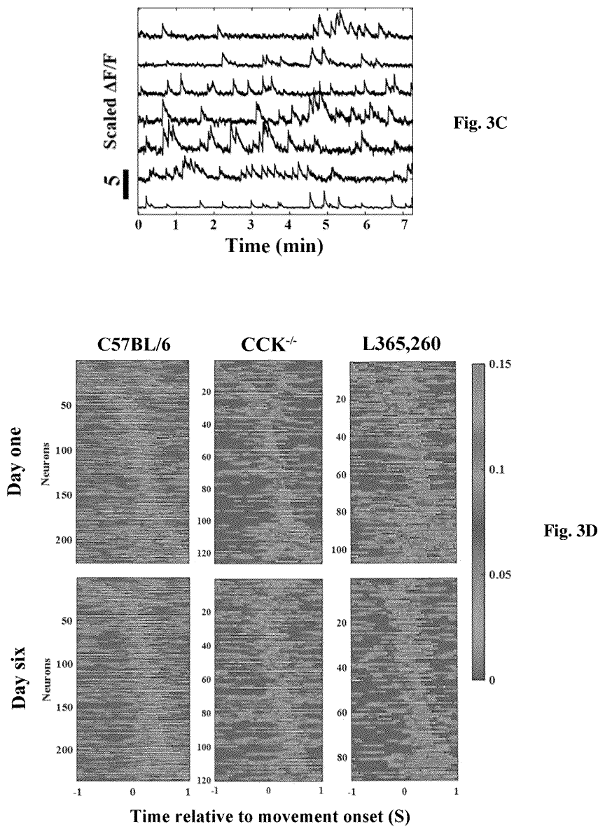

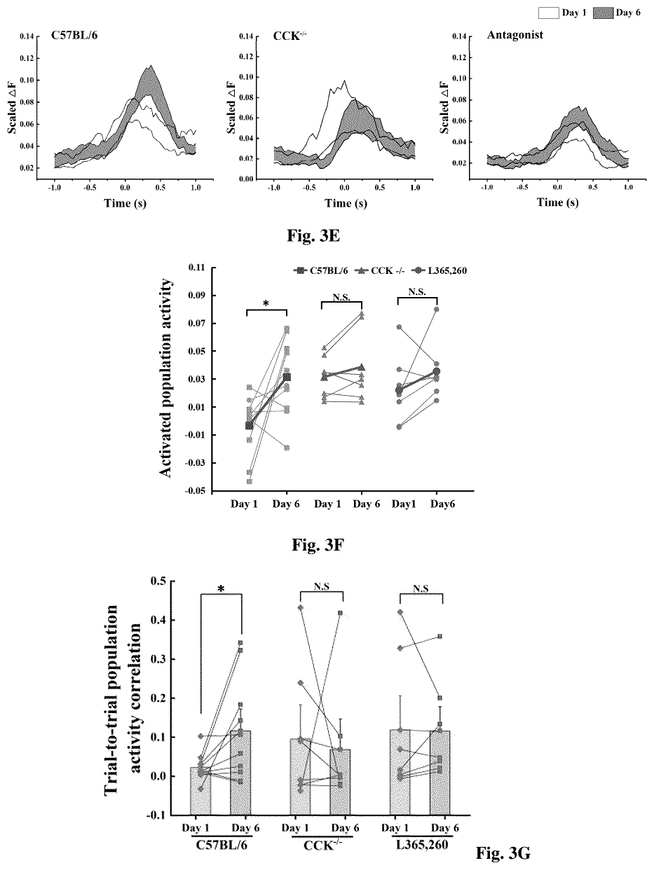

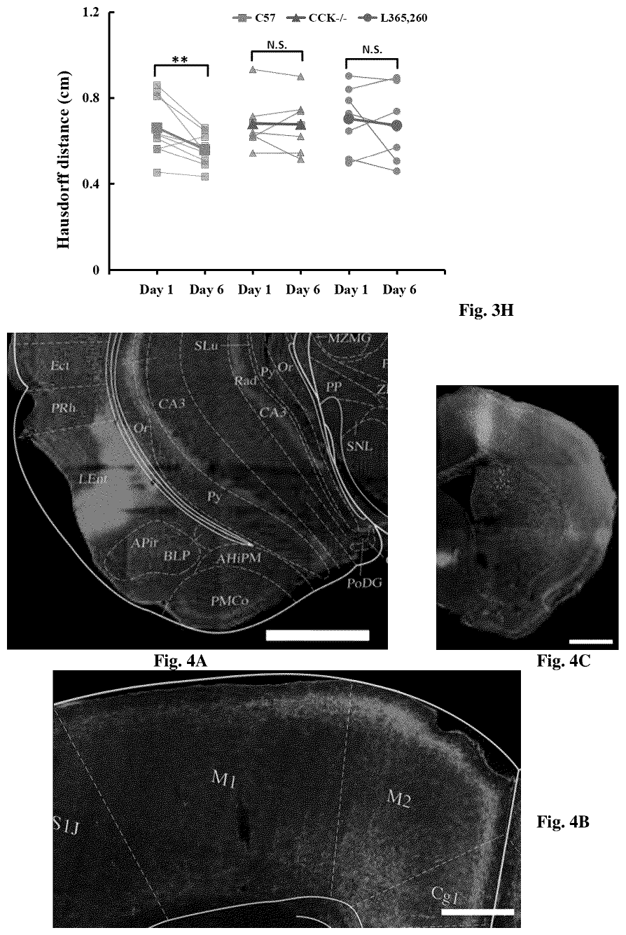

The inventors hypothesize that the CCK-enabled neuronal plasticity happens at the population level in the motor cortex. To test the hypothesis, a one-photon miniscope is attached over the motor cortex, contralateral to the dominant hand of the mouse, with an implanted high light transmission glass window in between (FIG. 3B). In particular, a wide-tip glass pipette tightly touches the brain surface by being lowered to a depth of 400-500 um below the original height of the brain surface, and strong GCaMP6s virus expression is observed in the superficial layer of the motor cortex with a high contrast compared with the deep layers after >14 days of expression. A baseplate is implanted on the skull, which is connected to the miniscope for calcium imaging during motor skills training (right panel in FIG. 3B). A web camera is installed in front of the training chamber to simultaneously monitor the mouse performing the task with the neuronal activities.

Three groups of mice are divided into: 1) C57BL/6, 2) CCK^{-/-}, and 3) C57BL/6 with CCKBR antagonist, to examine how CCK signaling affects neuronal activities in the motor cortex (FIG. 3A). GCaMP6s signals in layer 2/3 of the motor cortex is first confirmed, as shown in the examples (FIG. 3B). The neuronal signals are extracted with CNMF-E and analyzed with MATLAB (FIG. 3C). Neurons show various temporal and spatial responses to the movements during the task.

The neuronal activity pattern, excluding the indiscriminate neurons (ranksum test, neuronal activity during reaching & not reaching, p≥0.05), in the C57BL6 group, is refined after six days of training; the peak activity of the neurons become stronger with lower background activity (FIG. 3D). These results are similar to that of layer 2/3 neurons of the motor cortex in a mouse performing a lever-press task⁷. In contrast, the inventors found no apparent changes after training for six days for groups of CCK^{-/-} and C57BL/6 mice injected with the antagonist, the neuronal activity pattern (FIG. 3D).

The population activity of neurons varies with time relative to movement onset, starting to rise around 0.2 s before movement onset and reaching the peak at the time of 0.33 s after movement onset (FIG. 3E). The activated population activity, peak activity minus baseline activity, for C57BL/6 mice increases significantly with training (FIG. 3F; paired t-test, Day 1 vs. Day 6, 0.0216±0.0062 vs. 0.0593±0.0114, p=0.044<0.05). However, no significant change in the activated population activity is observed for both CCK^{-/-} and L365,260 groups (FIG. 3F; paired t-test, CCK^{-/-}, Day 1 vs. Day 6, 0.0313±0.0057 vs. 0.0386=0.0099, p=0.237; L365,260, Day 1 vs. Day 6, 0.0218±0.0094 vs. 0.0354±0.0080, p=0.240).

The Pearson correlation coefficient is adopted to evaluate the recurrence of neuronal activities among reaching trials. The average correlation coefficient of neuronal activities of different trials between Day 1 and Day 6 are compared. A significant increase in the trial-to-trial population activity correlation on Day 6 compared with Day 1 in the C57BL/6 mice group is observed (FIG. 3G, one-way RMANOVA, Day 1 vs. Day 6, 0.023±0.01 vs. 0.12±0.04, F[1,9]=5.342, p=0.046<0.05). However, no significant differences in the correlations between Day 1 and Day 6 are observed in the CCK-group, nor in the L365,260 group (FIG. 3G; one-way RM ANOVA, CCK^{-/-}, Day 1 vs. Day 6, 0.10±0.07 vs. 0.07±0.06, F[1,6]=0.073, p=0.796; L365/260, Day 1 vs. Day 6, 0.12±0.07 vs. 0.12±0.05, F[1,6]=0.005, p=0.944). The pairwise Hausdorff distance of trajectories in C57BL/6 group decreases significantly with training, while no significant changes are observed in CCK^{-/-} or L365,260 injection group, suggesting that the population activities are in line with the changes of the variation of the trajectories during motor learning (FIG. 3H; paired t-test, C57BL/6, Day 1 vs. Day 6, 0.6613±0.017 cm vs. 0.5588±0.0227 cm, p=0.0075<0.01; CCK^{-/-}, Day 1 vs. Day 6, 0.6787±0.0470 cm vs. 0.6760±0.0501 cm, p=0.9219; L365,260, Day 1 vs. Day 6, 0.7012±0.0594 cm vs. 0.6712±0.0659 cm, p=0.5606). The trial-to-trial population activity correlation in L365,260 group on Day 1 appears to be higher than that in C57BL/6 group. This might be due to that the drug blocked the trial-to-trial learning on Day 1, suppressing the exploration of the optimal path and abandonment of bad movements that would otherwise occur in wildtype mice.

Taken together, CCK deficiency causes defects in neuronal refinement and the reproducibility of neuronal activity among different trials during motor skill learning.

### CCK-Expressing Neurons in the Lateral Entorhinal Cortex Projecting to the Motor Cortex

Our next quest is to find what CCK projection is crucial in motor skill learning. It is known that CCK neurons in the entorhinal cortex, a gateway from the hippocampus to the neocortex, play critical roles in encoding sound-sound, visuoauditory, fear, and spatial memory^{13, 9, 14, 15}. These findings prompted us to examine whether CCK-expressing neurons in the entorhinal cortex also project to the motor cortex.

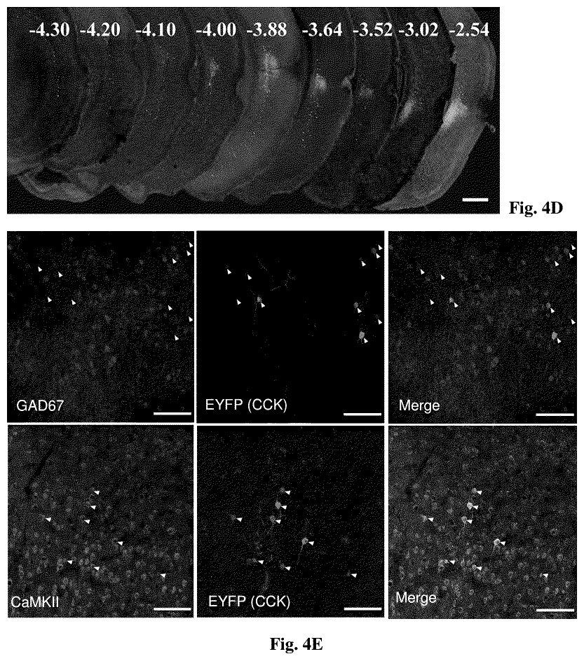

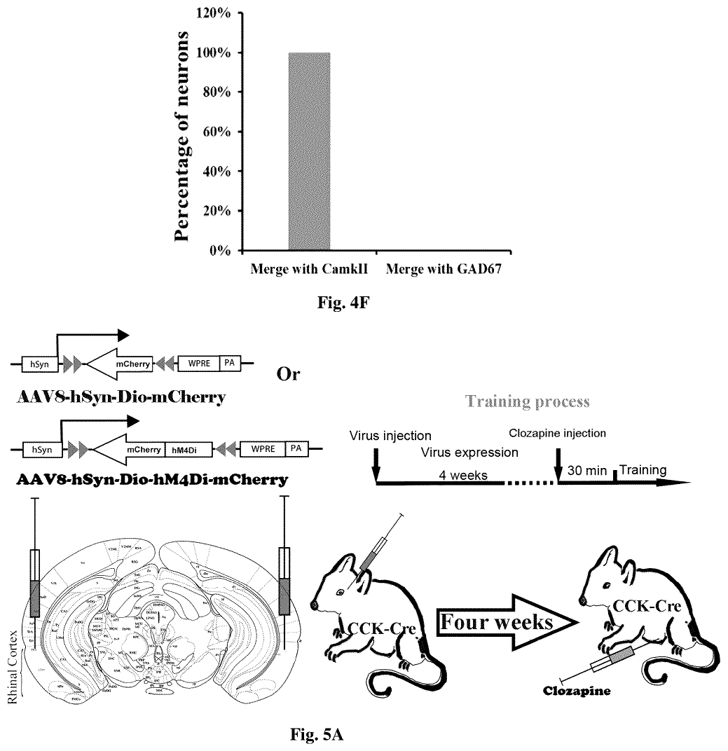

Both anterograde and retrograde viruses are used to track neuronal projections in this study. First, a Cre-dependent, highly efficient AAV virus expressing mCherry is injected into the rhinal cortex of one hemisphere of 8-week-old CCK Cre mice (FIG. 4A). This viral vector is expected to be taken up in the soma of neurons and transported to the axon terminus. In the motor cortex, mCherry-expressing neuronal axons mainly spread in layer 2/3 or layer 6 (FIG. 4B). Next, a Cre-dependent retrograde AAV vector expressing EYFP fluorescent protein gene is injected into the motor cortex in deep layers and superficial layers to verify the projections from the lateral entorhinal cortex to the motor cortex (FIG. 4C). In the rhinal cortex, the EYPF-labeled soma spread from AP: −2.54 to AP: −4.30, and local clusters are observed in layer 4 and layer 5, where the neurons are expected to project to the neocortex (FIG. 4D). Both anterograde and retrograde tracking results indicate that CCK-expressing neurons in the rhinal cortex projecting to the motor cortex are asymmetric, showing a preference for the ipsilateral hemisphere. Primary antibodies against GAD67 and CaMK2a are used for the immunostaining of the rhinal cortex sections to determine the characteristics of CCK neurons projecting to the motor cortex. None of the retrograde EYFP-labeled neurons merges with GAD67 staining but completely colocalize with CaMK2a staining, indicated by the white arrowhead, suggesting that the neurons projecting to the motor cortex are all excitatory neurons (FIG. 4E and FIG. 4F). Therefore, CCK neurons in the rhinal cortex may affect motor skill learning by regulating the plasticity of neurons in the motor cortex. FIGS. 4A-4F shows the labeling of CCK neuron projections from the RC to the MC. The scale bars represent 1000 um in FIGS. 4A, 4B, 4C, and 4D and 100 um in FIG. 4E.

### Inhibiting CCK Neurons in the EC/PC Suppresses Motor Learning

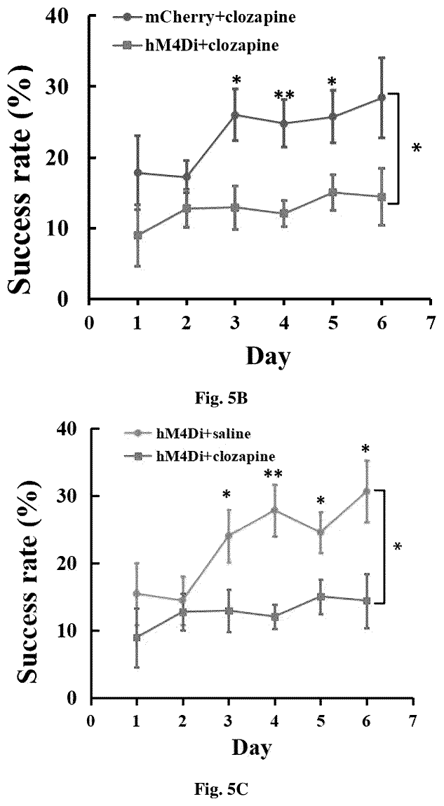

In the following experiment, chemogenetics are adopted to selectively silence the CCK projection neurons from the rhinal cortex to the motor cortex to examine their involvement in motor skill learning. A Cre-dependent AAV vector carrying hM4Di or mCherry is injected into the rhinal cortex bilaterally in CCK-Cre mice one month before the behavior test (FIG. 5A). Clozapine is intraperitoneally injected, followed by an approximately 30 min period for drugs to be taken up and transported to the brain. The drug binds to the hM4Di and inactivate the neurons (FIG. 5A). The success rate of hM4Di with the clozapine injection group shows no significant increase after six days of training, while the success rate of the control group of mCherry with clozapine injection increases significantly beginning on the third day of training and remains at a high level until the end of training (FIG. 5B; hM4Di+Clozapine group, one-way RM ANOVA, F[5,50]=0.839, p=0.528; mCherry+Clozapine group, one-way RM ANOVA, F[5,35]=3.121, p=0.02<0.05; two-way mixed ANOVA, post hoc. comparison between two groups, F[1,17]=7.014, p=0.016<0.05, hM4Di vs. mCherry, Day 3, 12.92%±3.10% vs. 25.99%±3.62%, F[1,17]=7.510, p=0.014<0.05; Day 4, 12.04%±1.84% vs. 24.78%±3.34%, F[1,17]=12.804, p=0.002<0.01; Day 5, 15.02%±2.55% vs. 25.74%±3.72%, F[1,17]=6.061, p=0.025<0.05; Day 6, 14.41%±4.01% vs. 28.42%±5.64%, F[1,17]=4.354, p=0.052.).

To exclude the possibility that hM4Di alone might regulate the neurons in this system, saline is administered as the control to the mice with the same virus vector with hM4Di injected into the rhinal cortex of CCK-Cre mice, as compared to the clozapine-administered experimental group (FIG. 5A). The learning curve of the control group injected with saline shows a learning trend in the single pellet reaching task, similar to the “mCherry+clozapine” group, and the success rate is significantly different from the “hM4Di+clozapine” group (FIG. 5C; hM4Di+saline group, one-way RM ANOVA, F[5,45]=7.911, p<0.001; between groups, two-way mixed ANOVA, significant interaction, F[5,95]=2.813, p=0.021<0.05, hM4Di+saline vs. hM4Di+clozapne, post hoc. comparison between two groups, F[1,19]=6.193, p=0.022<0.05; post hoc. comparison between two groups on different days, Day 3, 24.02%±3.93% vs. 12.12%±3.10%, F[1,19]=5.013, p=0.0373<0.05; Day 4, 27.81%±3.84% vs. 12.04%±1.84%, F[1,19]=14.534, p=0.0012<0.01; Day 5, 24.54%±3.05% vs. 15.02%±2.55%, F[1,19]=5.785, p=0.0263<0.05; Day 6, 30.60%±4.59% vs. 14.41±4.01%, F[1,19]=7.128, p=0.0151<0.05; The hM4Di+clozapine curve in FIG. 5C shared that in FIG. 5B). These results conclude that CCK neurons in the rhinal cortex may be crucial for motor learning.

Rescue of the Motor Learning Ability of the CCK Mice with CCK4

So far, the potential involvement of CCK in motor skill learning has been examined with several loss-of-function studies. The inventor next designs a gain-of-function experiment to see whether CCKBR agonist could rescue the defective motor learning ability. A tetrapeptide, CCK4 (Trp-Met-Asp-Phe-NH₂), a CCKBR agonist that can pass through the brain-blood barrier, is chosen to regain the defective motor learning ability of CCK^{-/-} mice¹⁴.

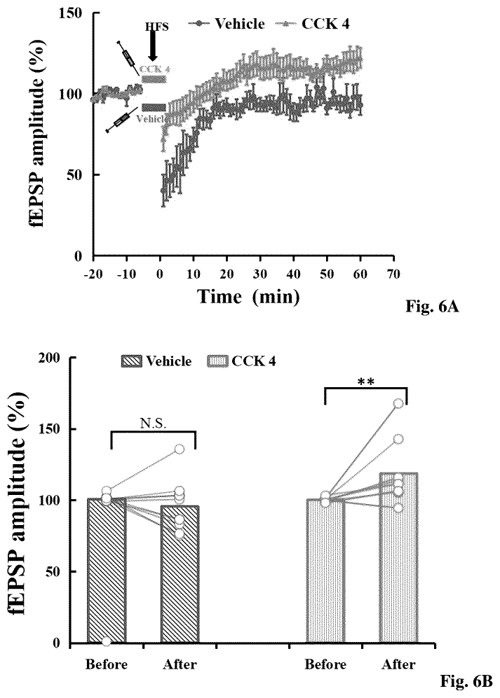

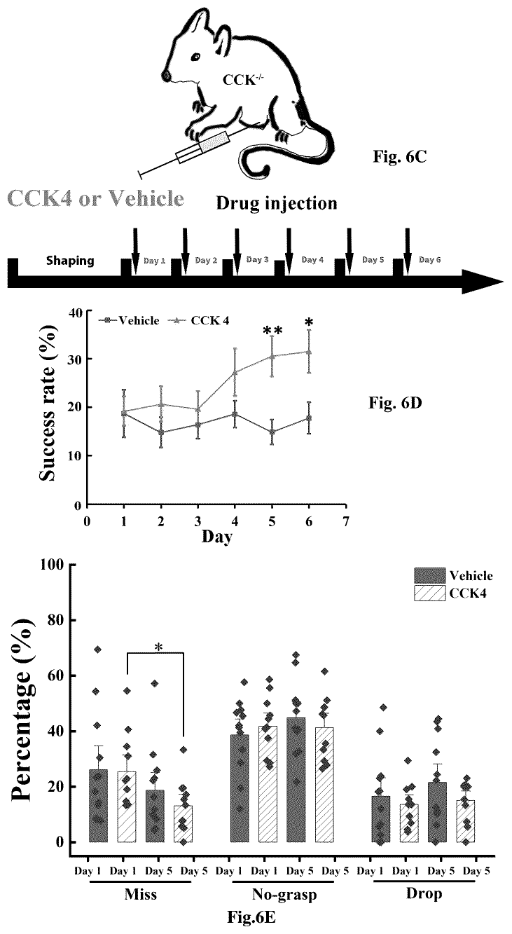

Firstly, the inventors examined whether CCK4 could rescue the defective neuronal plasticity in the motor cortex of CCK^{-/-} mice. Motor cortex electrophysiology recording is carried out on brain slices from the CCK^{-/-} mice. After 15 minutes of stable baseline recording, either the CCK4 or the vehicle control (0.9% saline solution with 0.1% DMSO) is injected into the electrode dish and applied HFS, followed by 60 minutes of recording. A significant rescuing effect is observed by CCK4 application before the HFS compared with its vehicle control (FIG. 6A and FIG. 6B; Vehicle vs. CCK4, two-way mixed ANOVA, significant interaction during −10 −0 min and 50-60 min, F[1,21]=10.656, p=0.004<0.01; post hoc. Comparison between two groups, F[1,21]=7.997, p=0.01<0.05; Vehicle, before vs. after, 100.95%±0.67% vs. 95.53%±5.77%, F[1,10]=1.239, p=0.292; CCK4, before vs. after, 100.28%±0.47% vs. 118.89%±6.09%, F[1,11]=11.653, p=0.006<0.01).

Next, the inventors investigated whether the CCK4 application could rescue the motor skill learning of CCK^{-/-} mice is examined. The CCK4 or vehicle solution is injected intraperitoneally to CCK^{-/-} mice every day before the 6-day training (FIG. 6C). The success rate of CCK4-injected group keeps at the baseline level in the first three days and starts to increase gradually from Day 4 to Day 6 (FIG. 6D; CCK4, one-way RM ANOVA, F[5,50]=3.914, p=0.005<0.01; Day 5 vs. Day 1, 30.58%±4.18% vs. 19.17%±3.03%, F[1,10]=5.680, p=0.038<0.05; Day 6 vs. Day 1, 31.50%±4.43% vs. 19.17%±3.03%, F[1,10]=6.893, p=0.025<0.05). In contrast, no improvement in the success rate is observed in the vehicle control group mice (FIG. 6D; Vehicle, one-way RM ANOVA, F[5,55]=0.476, p=0.793). The between-group comparison shows that the CCK4 group has significantly higher success rate from Day 5 to Day 6 compared to the vehicle group (FIG. 6D; Vehicle vs. CCK4, two-way mixed ANOVA, significant interaction, F[5,105]=2.405, p=0.043<0.05; post hoc. comparison between Vehicle and CCK4, Day 5, 14.88%±2.61% vs. 30.51%±4.18%, F[1,21]=10.459, p=0.004<0.01; Day 6, 17.76%±3.25% vs. 31.50%±4.43%; F[1,21]=6.412, p=0.019<0.05).

The inventors compared the detailed reaching results on Day 1 and Day 5 between the CCK4 and the vehicle groups. The present application found that the miss rate of the CCK4 group drops significantly at Day 5 compared to Day 1, while that of the vehicle group shows no significant change (FIG. 6E; paired t-test, Vehicle, Day 1 vs. Day 5, 26.12%±5.71% vs. 18.71%±4.31%; F[1,11]=1.155, p=0.305; CCK4, Day 1 vs. Day 5, 25.47%±4.03% vs. 13.13%±2.80, F[1,10]=6.643, p=0.028<0.05), suggesting that the CCK4 rescued the aiming in reaching. This result demonstrates that CCK4 could cross the brain-blood barrier and partially rescue the motor learning ability of CCK-mice (FIG. 6E).

Therefore, without intending to be limited by theory, it is believed that CCK is the crucial signal that enables motor leaning. Intraperitoneal injection of CCK4 is sufficient to rescue the motor learning ability by turning on the neuronal plasticity of the CCK^{-/-} mice.

### Discussion

CCK^{-/-} mice show defective motor learning ability, of which the success rate of retrieving reward remains at the baseline level compared to the wildtype mice with a significantly increased success rate. No LTP is induced by HFS in the motor cortex of CCK^{-/-} mice but readily in their wildtype control, indicating a possible association between the motor learning deficiency and neuronal plasticity in the motor cortex. In vivo calcium imaging demonstrates that the deficiency of CCK signaling may led to the defect in the population neuronal plasticity in the motor cortex affecting motor skill learning.

We found that the CCK-positive neurons in the rhinal cortex project to the motor cortex, using both anterograde and retrograde tracing methods. Inactivating the CCK neurons in the rhinal cortex using chemogenetic methods significantly suppresses the motor learning ability. Our further gain-of-function study reveals that intraperitoneal application of CCK4 can rescue the defective motor skill learning of CCK^{-/-} mice.

Neuronal plasticity of the motor cortex has been assessed by many researchers using multiple methods, such as single pellet reaching task and lever-press task^{1,7}. Other brain areas are also involved in motor skills learning, such as thalamus, striatum, cerebellum, and midbrain. Thalamocortical projections in the motor cortex are widely distributed in all layers, including inputs to corticospinal neurons in layer 5¹⁶. With single pellet reaching task training, thalamocortical neurons are biased in activating the corticospinal neurons that control the performance of the task, though the unbiased activation of corticospinal neurons is observed before training, suggesting that the thalamus selectively activates corticospinal neurons to generate better control of the forelimb movement with motor learning¹⁷. The spiking of Purkinje neurons switches from more autonomous, the baseline condition, to time-locked activation or silence before reaching onset to produce a state promoting a high quality of movement, as mice lean to direct a robotic manipulation toward a target zone¹⁸. The ventral tegmental area (VTA) dopaminergic projection in the motor cortex is necessary for motor skill learning but not for execution. The VTA projection to the motor cortex may facilitate the encoding of a motor skill memory by relaying food reward information related to the task¹⁹. As the core area where dexterous motor memory is encoded, the plasticity of the motor cortex enables animals to learn complex motor tasks.

CCK produced in the rhinal cortex has been identified as the key to transforming a paired tone into auditory memory in mice and rats by regulating the plasticity of neurons in the auditory cortex¹³. The present application finds that genetic knockout of the cck gene can cause defects in motor learning, while the success rate of wildtype mice increases to 30.94% on day 3. The success rate alone is not sufficient to describe the function of CCK in motor skill learning; therefore, the reaching result of the task is divided into four types, “miss”, “no-grasp”, “drop” and “success”. “Miss” is caused by defects in “aiming” and “advance”, indicating a low probability of hitting the pellet. Miss rate of the CCK^{-/-} mice decreases with learning but shows less improvement than the wildtype mice, suggesting that the brain areas controlled the “aiming” and “advance” are affected by CCK partially. Besides, “no-grasp” and the “success” rate of CCK″-mice remains at the same levels after training, but the “drop” rate increases, suggesting that the improved “miss” trials finally turned to “drop”. The variation of the trajectories of the CCK^{-/-} mice is lower than that of the wildtype mice on the first day, which is consistent with the previous results that the animals with low variation in trajectories learn worse than those with wide variation in trajectories at the initiation stage²⁰. The reason maybe that when animals perform a motor task, the wider the variation of the movement, the easier it is for the mice to find the best path to complete the task. The lack of CCK impairs the plasticity of neurons in the motor cortex, which is deemed the basis for motor learning.

The motor cortex plays the leading role in controlling motor memory encoding^{21, 22, 23, 24}. CCKBRs dominate CCKARs in the neocortex including the motor cortex^{25, 26}. Blockade of the CCKBRs in the motor cortex suppressed the improvement in the success rate of mice in the single pellet reaching task (FIG. 2B). The gradually improved success rate on Day 5 and 6 (FIG. 2B) after CCKBR antagonists could be due to the lasting of the antagonists not long enough to cover the whole training period, partially due to the diffusion of the antagonist. The performance of both the “antagonist” and “vehicle” groups is similar on the first day, indicating a similar neuronal baseline condition for each group. Activating CCKBR by CCK agonist improves motor skill learning.

Based on the evidence that CCK is important for neuronal plasticity of the motor cortex and motor skill learning, the next question is how CCK affects the changes in neuronal activity of the motor cortex during training. An earlier study finds that neuronal activities in layer 2/3 of the motor cortex are modified, exhibiting more reproducible spatiotemporal sequences of neuronal activities with motor learning⁷.

In the present invention, the neuronal activities related to the task in layer 2/3 of the motor cortex of C57BL/6 mice are refined with motor skill learning, the activation of neurons becoming more reproducible among trials. The reproducibility changes of neural activities are in parallel with the reduced variations in the trajectories of the C57BL/6 mice after training (FIG. 1G, FIG. 1H). However, CCK^{-/-} mice generate distinct changes in the neuronal activity in the motor cortex compared with C57BL/6 mice. The pattern of the peak activity and the trial-to-trial population correlation have no significant differences after six days of motor learning, suggesting no refinement in the neuronal circuit after motor learning in CCK^{-/-} mice (FIG. 3D).

In order to exclude a different background of neuronal activity due to long-term accommodation to the lack of CCK in CCK^{-/-} mice, the CCKBR antagonist, L365,260 is injected into the motor cortex of C57BL/6 mice and no significant changes are observed in the pattern of the peak activity and the trial-to-trial population correlation had after six days of motor learning, similarly to the CCK^{-/-} mice.

The entorhinal cortex is crucial for learning and memory^{27, 14}. Our group found that CCK is essential for neuronal plasticity in the auditory cortex¹³. In this research, we determined that CCK from the rhinal cortex may be critical for motor skill learning.

In the rhinal cortex, CCK-positive neurons that project to motor cortex are excitatory neurons (FIG. 4E, FIG. 4F). The roles of both CCK and glutamate in the neuronal plasticity and the relationship between CCK and glutamate have been studied before^{28, 9}. In the previous study, we found that CCK is critical for HFS induced LTP, and CCK release is triggered by the activation of (N-methyl-D-aspartate) NMDA receptors that could be located in the presynaptic membrane of CCK-positive neurons⁹. In the motor cortex, many CCK-positive neurons are GABAergic (y-aminobutyric acid) neurons, in which the role CCK played is not very clear. However, evidence shows that GABA may inhibit the release of CCK in the neocortex²⁹. Many glutamatergic neurons in the neocortex also express CCK³⁰. Further study in the future is needed to investigate the role of cortical CCK-positive neurons, including inhibitory and excitatory neurons, played in neuronal plasticity and motor skill learning.

The hippocampus system, including the rhinal cortex, plays an essential role in declarative learning based on the finding of the famous patient H.M.³¹. However, the understanding of the role of the hippocampus system in motor skills learning is not consistent. In the mirror tracking task, the performance of H.M. is on par with normal people, suggesting that the motor learning ability is not affected without the hippocampus system³¹. But in the other two motor learning tasks, rotary pursuit and bimanual tracking, the performance of H.M. is much worse than the control. Besides, the movement of H.M. is slower when performing the task. This explanation is not enough to exclude the effects of the hippocampus system on motor skill learning. Indeed, Corkin herself thought that the H.M. could perform tasks that required less demanding motor skills, but not the tasks demanding better motor skills^{31, 32}.

The single pellet reaching task is a complex and dexterous motor task requiring the neocortex and the whole motor system. Chemogenetic inactivation of CCK neurons in the rhinal cortex significantly impaired the mice's motor learning ability compared to the two control groups.

Based on the anterograde and retrograde tracing of the neurons in the rhinal cortex, projections terminals from the rhinal cortex to the motor cortex are distributed to the superficial and deep layers (FIG. 4B, FIG. 4D). Previous research on both layer 2/3 and layer 5 found that motor skill learning refined neuronal activity in layer 2/3 of the motor cortices of the mice in a lever-press task. Thus, the CCK projections in the superficial layer may be where plasticity occurs^{33, 34}. Two-photon calcium imaging results from previous research indicate that spine generation and elimination occurred in the apical dendrites (in the superficial layer) of neurons in layer 2/3. Still, the spines around the soma of the neurons in layer 2/3 show no significant changes³⁵, consistent with the location of CCK neuron terminals projecting from the rhinal cortex.

Therefore, CCK from the rhinal cortex may promote dexterous motor skill leaning by regulating the activity of the motor cortex of mice and presumably in other mammals such as, but not limited to humans.

### Rescuing Neuroplasticity and Motor Skill Learning

Our gain-of-function experiment by injecting CCK4 to rescue the defective learning ability of CCK-mice supports the critical role of CCK in neuronal plasticity of the motor cortex and motor skill learning of mice and presumably in other mammals such as, but not limited to humans. The CCKBRs of CCK^{-/-} mice are not influenced by knocking out the cck gene, making it possible that the exogenous CCK activates the CCKBRs¹⁴. CCK4, a tetrapeptide, can pass through the blood-brain barrier. CCK^{-/-} mice with the defective motor learning capability improved significantly after the daily, single intraperitoneal injection of CCK4, to a comparable level as their wildtype control at Day 5. The results of the rescuing experiment reveal a new target for facilitating motor rehabilitation.

### REFERENCE

1. Xu T, Yu X, Perlik A J, Tobin W F, Zweig J A, Tennant K, Zuo Y (2009), Rapid formation and selective stabilization of synapses for enduring motor memories, Nature 462 (7275), 915-919. https://doi.org/10.1038/nature08389.
2. Chen C C, Gilmore A, Zuo Y (2014), Study Motor skills learning by Single-pellet Reaching Tasks in Mice, J. Vis. Exp. (85), e51238. https://doi.org/10.3791/51238.
3. Zhu H, Roth B L (2015), DREADD: a chemogenetic GPCR signaling platform, Int. J. Neuropsychopharmacology 18 (1). https://doi.org/10.1093/ijnp/pyu007.
4. Tervo DGR, Hwang B Y, Viswanathan S, Gaj T, Lavzin M, Ritola K D, Karpova A Y (2016), A designer AAV variant permits efficient retrograde access to projection neurons, Neuron 92 (2), 372-382. https://doi.org/10.1016/j.neuron.2016.09.021.
5. Li X, Cao V Y, Zhang W, Mastwal S S, Liu Q, Otte S, Wang K H (2017), Skin suturing and cortical surface viral infusion improves imaging of neuronal ensemble activity with head-mounted miniature microscopes, J. Neurosci. Methods. 291, 238-248. https://doi.org/10.1016/j.jneumeth.2017.08.016.
6. Zhou P, Resendez S L, Rodriguez-Romaguera J, Jimenez J C, Neufeld S Q, Giovannucci A, Paninski L (2018), Efficient and accurate extraction of in vivo calcium signals from microendoscopic video data, Elife 7, e28728. https://doi.org/10.7554/eLife.28728.
7. Peters A J, Chen S X, Komiyama T (2014) Emergence of reproducible spatiotemporal activity during motor learning, Nature 510, 263-267. https://doi.org/10.1038/nature13235.
8. Wang X, Liu Y, Li X, Zhang Z, Yang H, Zhang Y, He Z (2017), Deconstruction of corticospinal circuits for goal-directed motor skills, Cell 171 (2), 440-455. https://doi.org/10.1016/j.cell.2017.08.014.
9. Chen X, Li X, Wong Y T, Zheng X, Wang H, Peng Y, He J (2019), Cholecystokinin release triggered by NMDA receptors produces LTP and sound-sound associative memory, Proc. Natl. Acad. Sci. U.S.A., 116 (13), 6397-6406. https://doi.org/10.1073/pnas.1816833116.
10. Javanmard M, Shlik J, Kennedy S H, Vaccarino F J, Houle S, Bradwejn J (1999), Neuroanatomic correlates of CCK-4-induced panic attacks in healthy humans: a comparison f two time points, Biol. Psychiatry 45 (7), 872-882. https://doi.org/10.1016/S0006-3223 (98) 00348-5.
11. Eser D, Leicht G, Lutz J, Wenninger S, Kirsch V, Schüle C, Mulert C (2009), Functional neuroanatomy of CCK-4-induced panic attacks in healthy volunteers, Hum. Brain Mapp. 30 (2), 511-522. https://doi.org/10.1002/hbm.20522.
12. Aydin O U, Taha A A, Hilbert A, Khalil A A, Galinovic I, Fiebach J B, Madai V I (2021) On the usage of average Hausdorff distance for segmentation performance assessment: hidden error when used for ranking, Euro. Radiol. Exp. 5 (1), 1-7. https://doi.org/10.1186/s41747-020-00200-2.
13. Li X, Yu K, Zhang Z, Sun W, Yang Z, Feng J, He J (2014), Cholecystokinin from the entorhinal cortex enables neural plasticity in the auditory cortex, Cell Res. 24 (3), 307-330. https://doi.org/10.1038/cr.2013.164.
14. Feng H, Su J, Fang W, Chen X, He J (2021), The entorhinal cortex modulates trace fear memory formation and neuroplasticity in the mouse lateral amygdala via cholecystokinin, Elife 10, e69333. https://doi.org/10.7554/eLife.69333.
15. Su J, Ye W, Liu H, Zhang B, Jendrichovsky P, Hoang A D, Chen X, Lau C Y G, Yu D, Xiong W, Tortorella M, He J (2019), Entorhinal cholecystokinin enables theta-burststimulation-induced hippocampal LTP and transfer of spatial memory, Neuron 10.2139/ssrn.3451411. https://doi.org/10.2139/ssrn.3451411.
16. Hooks B M, Mao T, Gutnisky D A, Yamawaki N, Svoboda K, Shepherd G M (2013), Organization of cortical and thalamic input to pyramidal neurons in mouse motor cortex, J. Neurosci. 33 (2), 748-760. https://doi.org/10.1523/JNEUROSCI.4338-12.2013.
17. Biane J S, Takashima Y, Scanziani M, Conner J M, Tuszynski M H (2016) Thalamocortical projections onto behaviorally relevant neurons exhibit plasticity during adult motor learning, Neuron 89 (6), 1173-1179. https://doi.org/10.1016/j.neuron.2016.02.001.
18. Wagner M J, Savall J, Hernandez O, Mel G, Inan H, Rumyantsev O, Schnitzer M J (2021), A neural circuit state change underlying skilled movements Cell 184 (14), 3731-3747. https://doi.org/10.1016/j.cell.2021.06.001.
19. Hosp J A, Pekanovic A, Rioult-Pedotti M S, Luft A R (2011), Dopaminergic projections from midbrain to primary motor cortex mediate motor skills learning, J. Neurosci. 31, 2481-2487. https://doi.org/10.1523/JNEUROSCI.5411-10.2011.
20. Wu H G, Miyamoto Y R, Castro LNG, Ölveczky B P, Smith M A (2014), Temporal structure of motor variability is dynamically regulated and predicts motor learning ability, Nat. Neurosci. 17 (2), 312-321. https://doi.org/10.1038/nn.3616.
21. Cheney P D (1985), Role of cerebral cortex in voluntary movements: A review, Physical Therapy 65 (5), 624-635. https://doi.org/10.1093/ptj/65.5.624.
22. Sanes J N, Donoghue J P (2000), Plasticity and primary motor cortex, Anmu. Rev. Neurosci. 23 (1), 393-415. https://www.annualreviews.org/doi/10.1146/annurev.neuro.23.1.393.
23. Economo M N, Viswanathan S, Tasic B, Bas E, Winnubst J, Menon V, Svoboda K (2018), Distinct descending motor cortex pathways and their roles in movement, Nature 563 (7729), 79-84. https://doi.org/10.1038/s41586-018-0642-9.
24. Svoboda K, Li N (2018), Neural mechanisms of movement planning: motor cortex and beyond, Churr: Opin. Neurobiol. 49, 33-41. https://doi.org/10.1016/j.conb.2017.10.023.
25. Crawley J N, Corwin R L (1994), Biological actions of cholecystokinin, Peptides 15 (4), 731-755.https://doi.org/10.1016/0196-9781 (94) 90104-X.
26. Wank S A (1995), Cholecystokinin receptors, Am. J. Physiol. 269 (5), G628-G646. https://doi.org/10.1152/ajpgi.1995.269.5.G628.
27. Chen X, Guo Y, Feng J, Liao Z, Li X, Wang H, He J (2013), Encoding and retrieval of artificial visuoauditory memory traces in the auditory cortex requires the entorhinal cortex, J. Neurosci. 33 (24), 9963-9974. https://doi.org/10.1523/JNEUROSCI.4078-12.2013.
28. Bandopadhyay R, Belleroche De J (1991) Regulation of CCK release in cerebralcortex by N-methyl-D-aspartate receptors: Sensitivity to APV, M K-801, kynurenate, magnesium and zinc lons, Neuropeptides 18 (3), 159-163. https://doi.org/10.1016/0143-4179 (91) 90108-U.
29. Yaksh T L, Furui T, Kanawati I S, Go VLW (1987), Release of cholecystokinin fromrat cerebral cortex in vivo: role of GABA and glutamate receptor systems, Brain Res. 406 (1-2), 207-214. https://doi.org/10.1016/0006-8993 (87) 90784-0.
30. Watakabe A, Hirokawa J, Ichinohe N, Ohsawa S, Kaneko T, Rockland K S, YamamoriT (2012), Area-specific substratification of deep layer neurons in the rat cortex, J. Comp. Neuro. 520 (16), 3553-3573. https://doi.org/10.1002/cne.23160.
31. Corkin S (1968), Acquisition of motor skill after bilateral medial temporal-lobe excision, Neuropsychologia 6 (3), 255-265. https://doi.org/10.1016/0028-3932 (68) 90024-9.
32. Brigard De F (2019) Know-how, intellectualism, and memory systems, Philos. Psychol. 32 (5), 719-758. https://doi.org/10.1080/09515089.2019.1607280.
33. Peters A J, Lee J, Hedrick N G, ONeil K, Komiyama T (2017), Reorganization of corticospinal output during motor learning, Nat. Neurosci. 20 (8), 1133-1141. https://doi.org/10.1038/nn.4596.
34. Heindorf M, Arber S, Keller G B (2018), Mouse motor cortex coordinates the behavioral response to unpredicted sensory feedback, Neuron 99 (5), 1040-1054. https://doi.org/10.1016/j.neuron.2018.07.046.
35. Chen S X, Kim A N, Peters A J, Komiyama T (2015), Subtype-specific plasticity of inhibitory circuits in motor cortex during motor learning, Nat. Neurosci. 18, 1109-1115. https://doi.org/10.1038/nn.4049.
36. Papale A E, Hooks B M (2018), Circuit changes in motor cortex during motor skills learning, Neurosci. Rev. 368, 283-297. https://doi.org/10.1016/j.neuroscience.2017.09.010.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A method for treating movement disorders comprising a step of administrating a CCK-B receptor agonist to a subject in need thereof.

2. The method of claim 1, wherein the movement disorders are caused by a condition selected from the group consisting of a brain injury, a stroke, Chorea, Dystonia, Parkinson's disease, brain infarction, neuron death or less of neuronal plasticity due to aging, and a combination thereof.

3. The method of claim 1, wherein the CCK-B receptor agonist is selected from the group consisting of CCK4, a CCK4 analogue, and a mixture thereof.

4. The method of claim 1, wherein the CCK-B receptor agonist is administrated via a route selected from the group consisting of intramuscular injection, intraperitoneal administration, intravenous administration, and a combination thereof.

5. The method of claim 1, wherein the CCK-B receptor agonist is administrated over a period of about one week to four weeks.

6. The method of claim 1, wherein the CCK-B receptor agonist is administrated at a dosage of about 0.0036 mg/kg to about 0.036 mg/kg.

7. The method of claim 1, wherein the CCK-B receptor agonist is effective in regaining motor learning ability in adulthood.

8. The method of claim 2, wherein the stroke is selected from the group consisting of an ischemic stroke, hemorrhagic stroke, cryptogenic stroke, and a combination thereof.

9. The method of claim 2, wherein the brain injury is an injury in motor cortex.

10. A method of regaining motor learning ability comprising a step of administrating a CCK-B receptor agonist to a subject in need thereof.

11. The method of claim 10, wherein the CCK-B receptor agonist is selected from the group consisting of CCK4, a CCK4 analogue, and a mixture thereof.

12. The method of claim 10, wherein the CCK-B receptor agonist is administrated via a route selected from the group consisting of intramuscular injection, intraperitoneal administration, intravenous administration, and a combination thereof.

13. The method of claim 10, wherein the CCK-B receptor agonist is administrated over a period of about one week to four weeks.

14. The method of claim 10, wherein the CCK-B receptor agonist is administrated at a dosage of about 0.0036 mg/kg to about 0.036 mg/kg.

15. The method of claim 10, wherein the CCK-B receptor enables neuronal plasticity in motor cortex.

16. The method of claim 10, wherein the CCK-B receptor is effective in regaining motor learning ability in adulthood.

17. Use of a CCK-B receptor agonist in the manufacture of a medicament for treating movement disorders by administrating it to a subject in need thereof.

18. The use of claim 17, wherein the CCK-B receptor agonist is selected from the group consisting of CCK4, a CCK4 analogue, and a mixture thereof.

19. The use of claim 17, wherein the CCK-B receptor agonist is in a dosage form suitable for an administration route selected from the group consisting of intramuscular injection, intraperitoneal administration, intravenous administration, and a combination thereof.

20. The use of claim 17, wherein the CCK-B receptor agonist is comprised in an amount of about 80% to about 90% by weight of the medicament.
